(19) United States
(12) Patent Application Publication
(10) Pub. No.: US 2016/0262929 A1
(43) Pub. Date: Sep. 15, 2016

(54) FreezePocket

(71) Applicant: Martha Teresa Rodao, Woburn, MA (US)

(72) Inventor: Martha Teresa Rodao, Woburn, MA (US)

(21) Appl. No.: 14/756,357

(22) Filed: Sep. 3, 2015

Related U.S. Application Data
(60) Provisional application No. 62/131,448, filed on Mar. 11, 2015.

(51) Int. Cl. A61F 7/10

(52) U.S. Cl. CPC A61F 7/10; A61F 2007/108; A61F 2007/105

(57) ABSTRACT

FreezePocket is a therapeutic device that secures plastic ice packs, allowing an individual to apply targeted cold temperature to multiple different injured areas of the body. The device functions in a hands-free manner and allows for re-application of treatment over an extended period of time, while the user rests comfortably.

### BACKGROUND OF THE INVENTION

Ice packs have been used for a long time. Their use in the healing process of torn muscles and connective tissue has been scrutinized by the practitioners of sports medicine in allowing the recuperation of injured athletes as quickly as possible.

Generally, icing an injury decreases pain and provides vasoconstriction (closing of small blood vessels). This helps limit the amount of swelling that occurs immediately after the injury. However, a person icing an injury usually needs to hold the ice pack with their hand, which can put the user at risk for frostbite. Therefore, there exists a need for an ice pack holder that allows the user to apply ice without using their hands. Devices are known that related to pouches for holding cold packs therein. Some devices provide a pouch for receiving re-usuable hot or cold packs which do include a strap for securing it to the body of the individual. FreezePocket uses several traditional plastic ice packs in a single pouch, which stays cold for an extended period of time. The pouch holder further includes a removable strap for encircling the ice pack holder around an individual. It is washable and could be stored in freezers along with plastic ice packs therein.

### SUMMARY OF THE INVENTION

The invention provides a holder for plastic ice packs. The ice packs holder includes a pouch having an interior volume divided in sections; it is made of cloth material, essentially rectangular in shape, and each pocket is configured to receive an elongated flap that allows the pouch to be covered. The pouch further includes an elongated strap that attaches to the pouch and allows a user to wrap the plastic ice pack holder around his limb or torso. Plastic ice packs stay cold for an extended period of time. Individuals can apply cold for 20 minutes every 3 to 4 hours, as directed by clinicians without the hassle of repeatedly going to the freezer because of the need to replace ice or replace a warm gel pack.

### BRIEF DESCRIPTION OF DRAWINGS

Referring now to FIGS. 1, 2, 3, 4, 5, 6, 7, 8, 9 these show front, back, side and overhead perspectives views of several preferred embodiments of the plastic ice pack holder.

FIG. 1 shows a front perspective view of a four pocket version of the present invention.

FIG. 2 shows a back perspective of the four pocket version of the present invention.

FIG. 3 shows a side view of the four pocket version of the present invention applied to a back.

FIG. 4 shows a front perspective of three pocket version of the present invention.

FIG. 5 shows a side/overhead perspective view of the three pocket version of the present invention.

FIG. 6 shows a front/overhead perspective view of the three pocket version of the present invention.

FIG. 7 shows a front perspective view of a three pocket version of the present invention with pockets now in a horizontal configuration.

FIG. 8 shows a front view of the present invention placed on the neck.

FIG. 9 shows a front perspective view of a two pocket version of the present invention.

### DETAILED DESCRIPTION OF INVENTION:

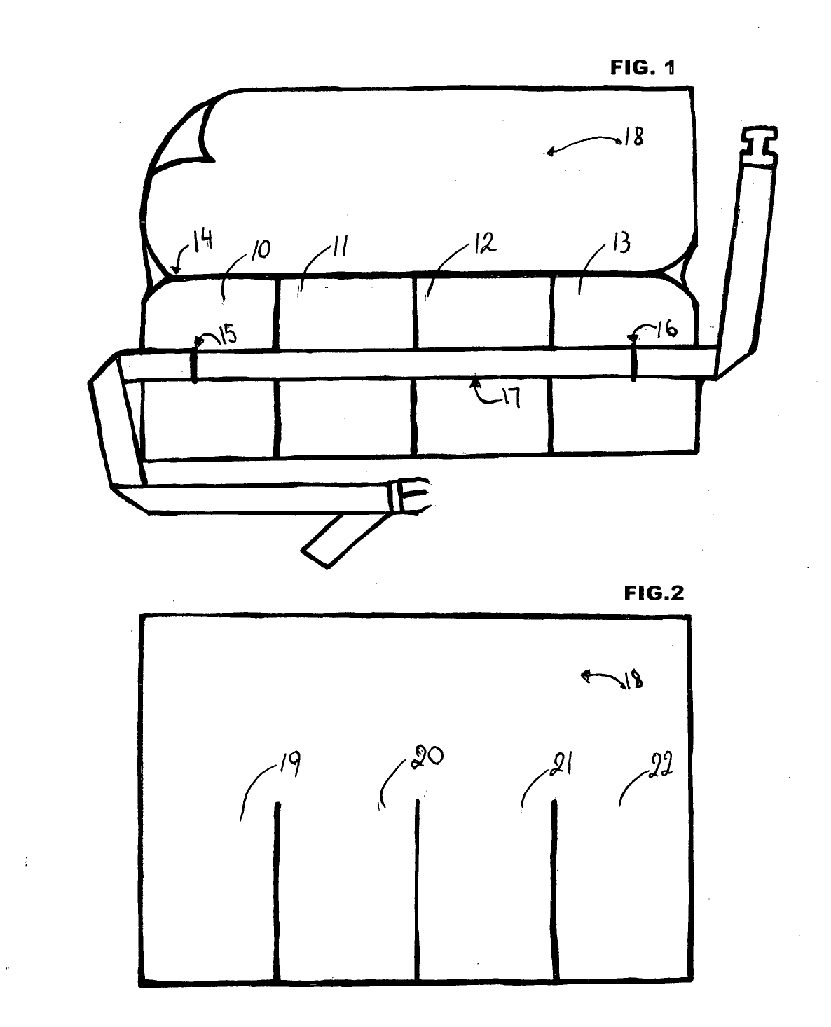

Referring to FIG. 1, there is shown a front perspective of the plastic ice pack holder, which can be made to hold small or large plastic ice packs. This holder or pouch is intended to be composed of two kinds of cloth material; a double flannel fabric that absorbs the moisture of the ice packs defrosting and a double nylon fabric which is a water repellent. However, any other material known in the art could be used. The plastic ice pack holder includes a pouch having the first end No.10 and second end No.13 forming an interior volume. The pouch is substantially U-shaped with the first end and second end at the ends of the U-shape; with dimensions slightly larger than the plastic ice packs to be used. The ice holder is one pouch, which subdivides the interior volume into compartments shown in FIG. 1 as Nos. 10, 11, 12, and 13. Each said compartment or pocket is configured to receive and enclose a plastic ice pack therein. Additionally, the ice packs are secured with an elastic No. 14, sewn across on the top and edge of the pockets. Two loops No. 15 and No. 16 are placed on the front of the first end No.10 and last end No. 13, preferably made of ribbon material. The design includes an elongated fastener, No 17 that is configured to allow the user to wrap the pouch around specific areas of his/her body, such as a limb or torso. This fastener is a removable strap that is slid through the loops, Nos. 15 and 16, and is configured to secure the plastic ice packs within each said pockets, applying pressure, to evenly disperse the cold across said pouch. The strap is preferably made of polypropylene webbing well known in the art, although any other related material could be used. Buckles or Velcro, easily obtainable commercially, are attached to both ends of said strap.

Referring to FIG. 2, there is shown the back of the above described pouch. Nos. 19, 20, 21, 22 show a seam dividing said pouch into four compartments matching those on FIG. 1 shown at Nos. 10, 11, 12, and 13, respectively forming the pockets. The remainder of the nylon fabric on top of the U-shape form a flap shown on FIG. 1 at No. 18. This is configured to either enclose the pouch covering each pocket, or in the alternative, folded backward it reduces the level of cold. The elastic shown on FIG. 1, No. 14, which is placed at the top edge of each pocket allowing the flap to easily fold due to the stretchy nature of materials.

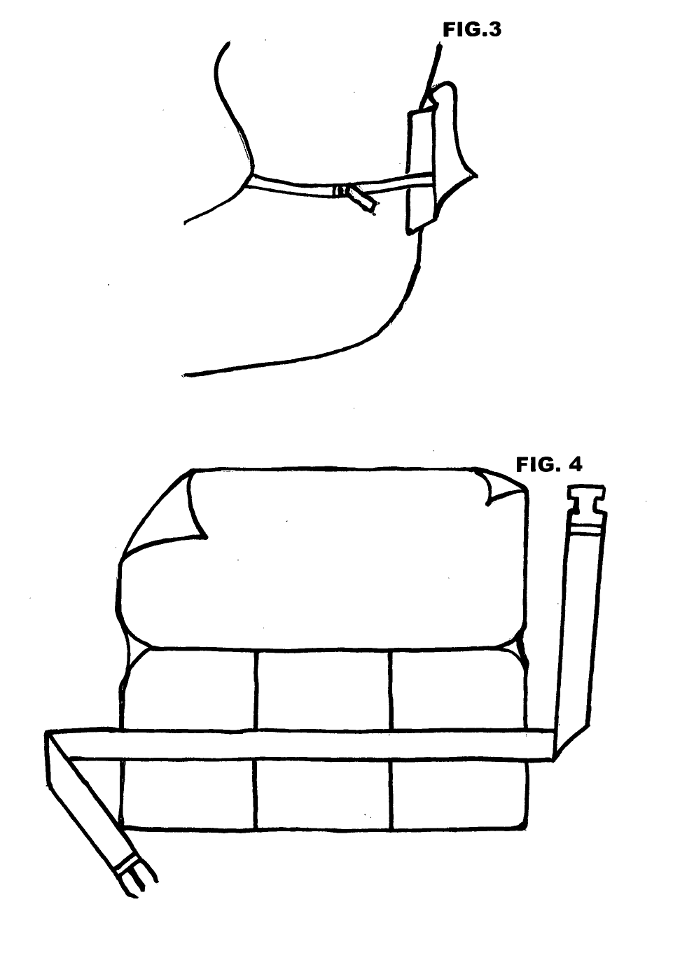

Referring to FIG. 3, there is shown an example of a side view of a 4 pocket large plastic ice packs holder of preferred embodiment placed at the lower back.

Referring to FIG. 4, there is shown an ice pack holder configured to receive 3 larger plastic ice packs therein. Its construction is basically similar to that shown in FIG. 1 and FIG. 2.

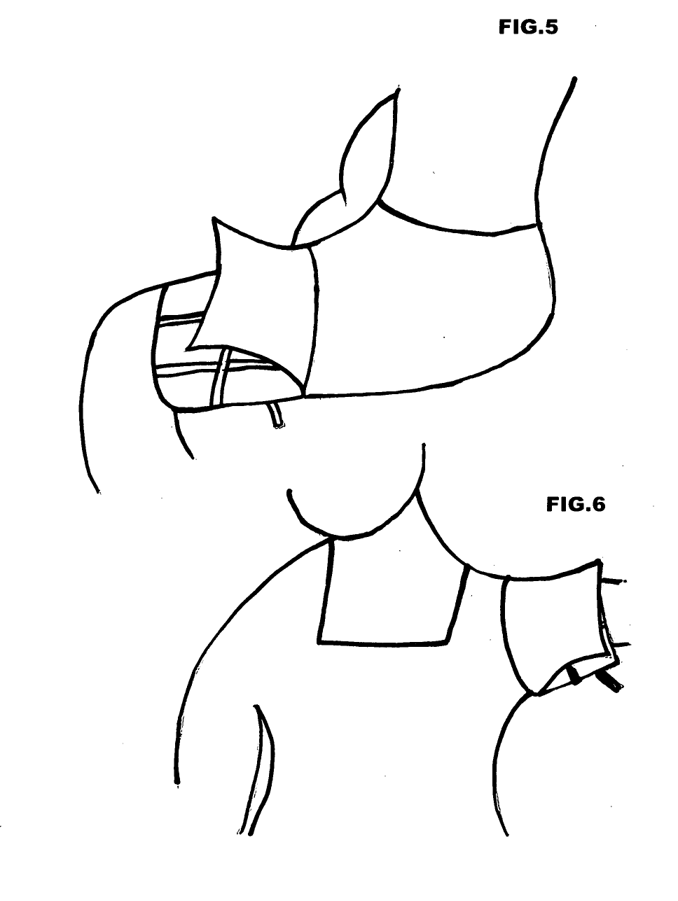

Referring to FIG. 5, there is shown an example of the application of a 3 large plastic ice pack holder to a knee area.

Referring to FIG. 6, there is shown a three pocket small plastic ice pack holder placed around a limb.

Referring to FIG. 7, there is shown an ice pack holder configured to receive 3 small plastic ice packs in horizontal position. While the construction is similar to that shown in FIGS. 1 and 2, instead of the strap, No. 17, there are two Velcro ribbons or tabs placed on the front of the first and last pockets of the holder. The placement is shown at No. 23 and No. 24.

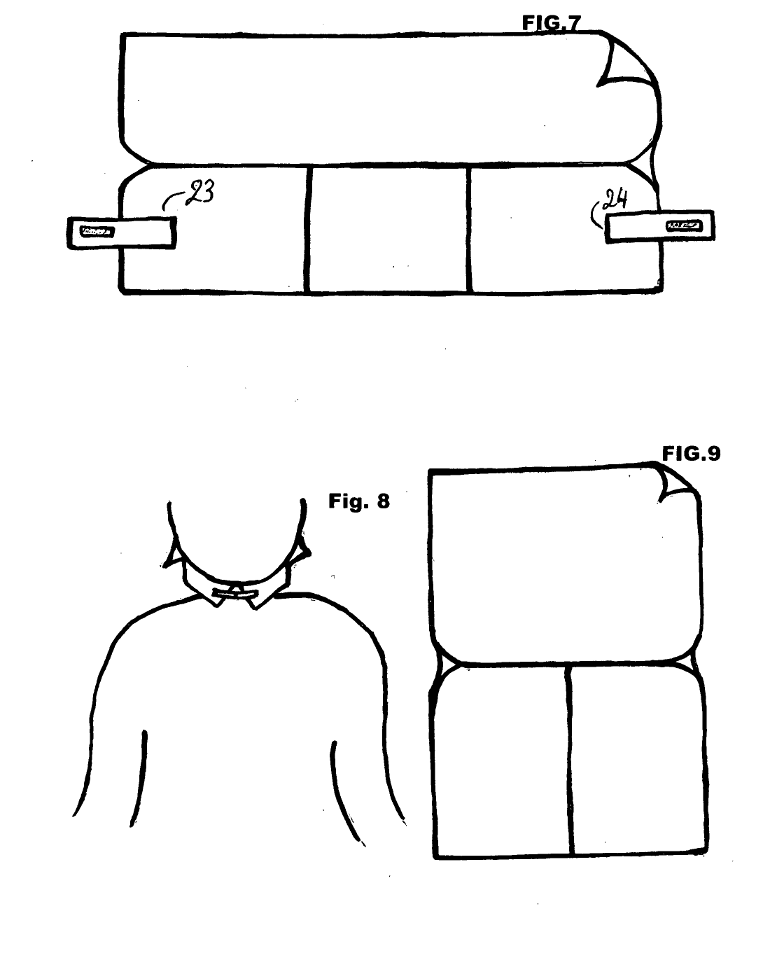

Referring to FIG. 8, there is shown an application at an individual's neck of the plastic ice pack holder configured to receive 3 small plastic ice packs therein in horizontal position as described in FIG. 7.

Referring to FIG. 9, there is shows a version of a plastic ice pack holder configured to receive two plastic ice packs. Its construction is similar to that shown in FIGS. 1 and 2.

The forgoing description of specific embodiments of the presented invention have been provided for purposes of description of design, and obviously many modifications are possibly in light of the above descriptions. The particular examples were chosen and described in order to best explain the principles of the present invention and its practical applications.

1. This invention allows for multiple reusable and commercial tradtional plastic ice packs to be placed in connected pockets in a single pouch or holder that can be applied to different injured areas of the body. It allows for repeated applications of cold without requiring the user to frequently get up to replace or re-freeze the ice packs. It allows the user to easily and safely repeat treatments over an extended period of time with a hands-free tool and offers a way for a user to rest comfortably while tending to soreness and injury. This is a unique economical invention that, as set forth below, fills an unmet need for a convenient, safe and durable device for extended icing treatments. Even though there are some devices available for holding cold packs with a pouch and strap, they lack the convenience and extended use period of the FreezePocket. They may require the user to buy multiple units, and using gel or water, they have a limited effective timeframe. The FreezePocket is a significant advance on these currently available devices. Among other advantages-, it fills the need for an ice packs holder with multiple pockets, eliminating the need to purchase multiple units to cover an affected area of the body. Furthermore, it is a device that allows for an extended effective use time eliminating the need for constant replacement. This allows an individual to rest comfortably even overnight while applying treatment to an injured area, because it stays frozen and/or cold for a long period time and its construction materials prevent wetness. Most importantly in this regard, it is safer since the individual does not have to return to the freezer for replacements during the night, or request the help of others on a frequent basis. The present invention provides a new application for the well-known and readily available plastic ice packs that are commonly used for cooling food in small coolers or lunch bags. They are easily available, inexpensive and are to be found in most any supermarket or convenience store in the United States. The object of this invention is to provide a device of low cost, significant safety and convenience. An individual can attend work, sports or other recreational activities and at the same time as keeping their water cold or food fresh with their plastic ice packs they can, when convenient, attend to an injury with their FreezePocket plastic ice pack holder. FreezePocket is so versatile it allows the individual to continue with his or her activity or work or to simply rest quietly at home while being able to conveniently and safely treat an injury or pain.
